(19) United States
(12) Patent Application Publication
(10) Pub. No.: US 2025/0149162 A1
(43) Pub. Date: May 8, 2025

(54) THREE-DIMENSIONAL HOLOGRAM PATIENT ENGAGEMENT SYSTEM

(71) Applicant: Steve Sterling, Key Biscayne, FL (US)

(72) Inventor: Steve Sterling, Key Biscayne, FL (US)

(21) Appl. No.: 18/940,695

(22) Filed: Nov. 7, 2024

Related U.S. Application Data
(60) Provisional application No. 63/547,642, filed on Nov. 7, 2023.

(51) Int. Cl. G16H 40/67; G03H 1/00; G16H 80/00

(52) U.S. Cl. CPC G16H 40/67; G03H 1/0005; G16H 80/00; G03H 2210/30

(57) ABSTRACT

A hologram patient engagement system allows patients to meet and engage with general practitioners, prescribers, medical specialists, and other healthcare professionals in life-like 3D at convenient locations that are remote from these healthcare providers. The system includes a 3D hologram display unit that can be networked between one or more locations with encrypted connectivity for security and privacy throughout the network. The system provides universal device-compatible integration with all wireless or wired patient diagnostic devices or remote patient monitoring devices via USB, HDMI or other forms of connectivity. The hologram display device is equipped with a camera, integrated stereo speakers, a touch screen control with integrated software and provides self-contained plug and play convenience.

This non-provisional patent application is based on provisional patent application Ser. No. 63/547,642 filed on Nov. 7, 2023.

### BACKGROUND OF THE INVENTION

### Field of the Invention

The present invention relates to remote visual and audio communication devices and, more particularly, to a 3D hologram patient engagement system including a 3D hologram display that allows patient engagement with general practitioners, prescribers, medical specialists and other healthcare professionals at convenient remote locations.

### Description of the Related Art

In the healthcare field, it is not always practical or even possible for general practitioners, doctors, physician assistants, nurses or other medical specialists to be physically present at the same location as a patient. For example, a doctor or other medical specialist may operate in a medical practice that covers a large geographical area and it may not be possible for the doctor or medical specialist to be at the same location as a patient on a particular appointment day. Additionally, there is a growing need for enhanced patient-doctor engagement at lower costs while offering a more personal experience for patients whether in a clinic, hospital, urgent care, pharmacy, or doctor shortage areas or other remote locations.

### SUMMARY OF THE INVENTION

The 3D hologram patient engagement system of the present invention offers patients the ability to meet and engage with general practitioners, prescribers, medical specialists, and other healthcare professionals at a convenient remote location, while providing a life-like 3D personal interaction experience between the patient and healthcare professional.

More particularly, the patient engagement system of the present invention includes a 3D hologram display unit that can be networked between one or more locations with encrypted connectivity for security and privacy throughout the network. The system provides universal device-compatible integration with all wireless or wired patient examination and diagnostic devices, as well as patient monitoring devices, via USB, HDMI, Ethernet, WiFi or other forms of connectivity. Examples of patient diagnostic devices that can be connected to the 3D hologram patient engagement system include, but are not limited to, monitors (e.g., vitals monitors), handheld dermascopes, iris scopes, an ORUS scope kit, a three-in-one diagnostics set, handheld otoscopes, and handheld general examination scopes and other devices.

### BRIEF DESCRIPTION OF THE DRAWINGS

For a fuller understanding of the nature of the present invention, reference should be had to the following detailed description taken in connection with the accompanying drawings in which:

FIG. 1A is a front perspective view of the 3D hologram display device in accordance with one embodiment of the present invention and illustrating the several components and features thereof;

FIG. 1B is a front elevational view of the 3D hologram display device in accordance with another embodiment of the present invention and illustrating the several components and features thereof;

FIG. 2A illustrates an all-in-one vitals monitor;

FIG. 2B is a perspective view illustrating a handheld dermascope;

FIG. 2C is a perspective view illustrating an iris scope;

FIG. 2D is a perspective view of an ORUS scope kit;

FIG. 2E is a perspective view of a three-in-one diagnostics set;

FIG. 2F is a perspective view illustrating a handheld otoscope;

FIG. 2G is a perspective view illustrating a handheld general examination scope; and

FIG. 3 is a schematic illustration showing a variety of connector ports providing universal device compatible integration with all wired and wireless patient examination, diagnostic, and monitoring devices into a connectivity panel that includes, but is not limited to, HDMI, USB, Ethernet and wireless (WiFi) or other means of wireless transmission.

Like reference numerals refer to like parts throughout the several views of the drawings.

### DETAILED DESCRIPTION OF THE PREFERRED EMBODIMENT

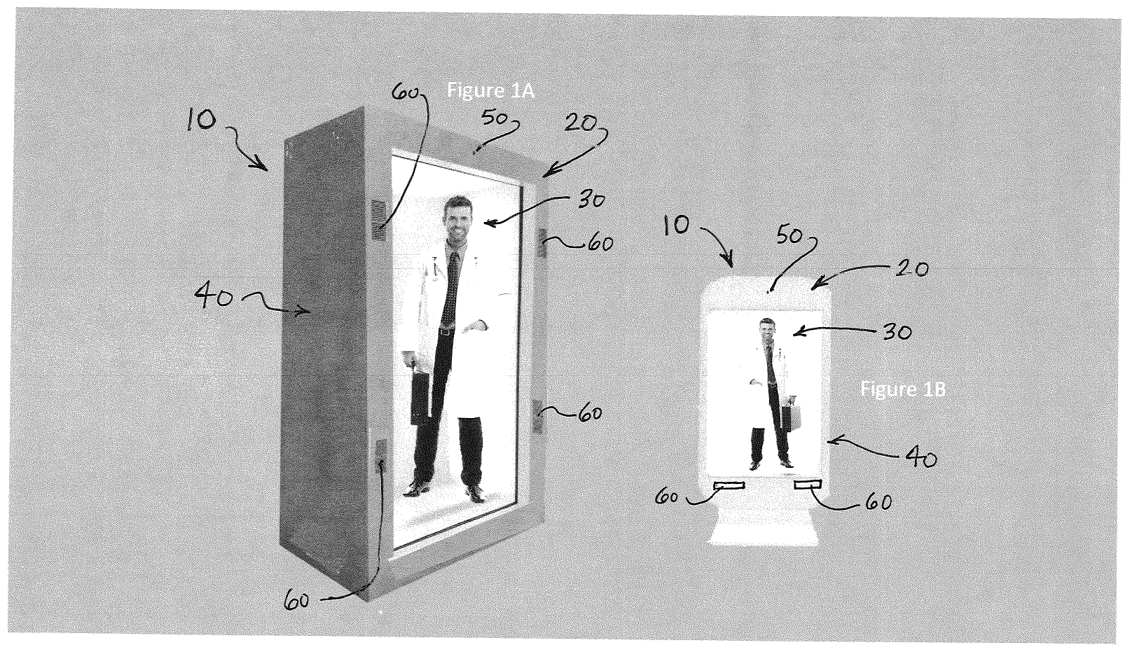

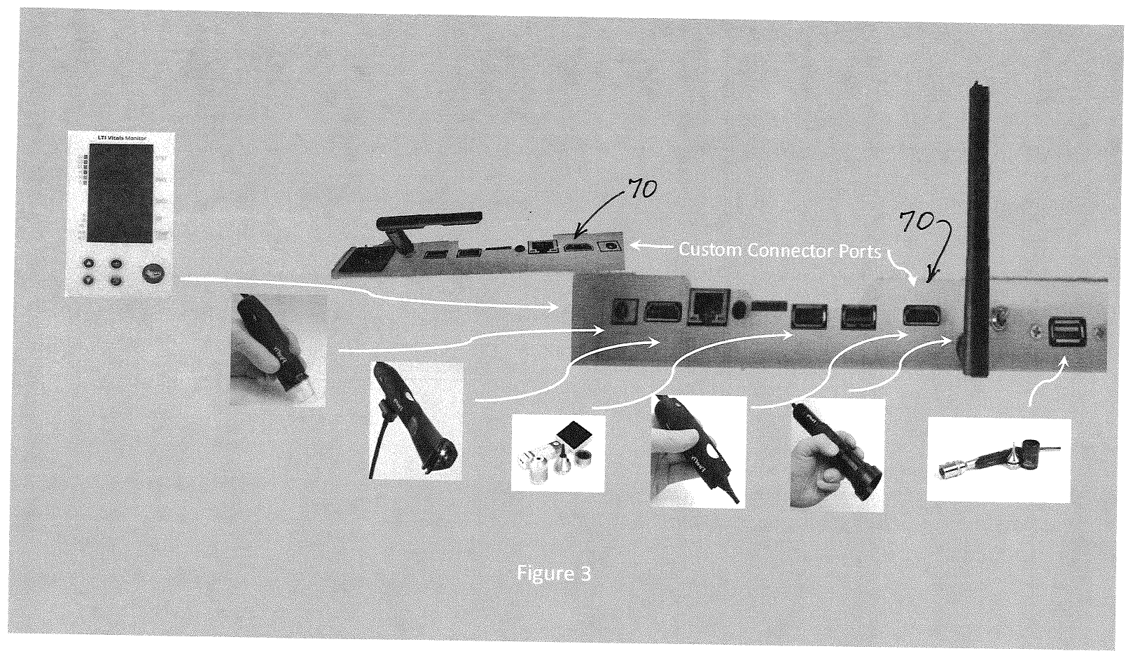

The present invention is directed to a 3D hologram patient engagement system, and is generally indicated as 10 in FIGS. 1A and 1B. Referring initially to FIGS. 1A and 1B, the system includes a 3D hologram display device 20 that is devised to facilitate enhanced patient-doctor engagement at lower costs, while offering a more personal experience for patients whether in a clinic, hospital, urgent care, pharmacy, doctor shortage areas or other remote locations. The 3D hologram display device 20 includes a 3D hologram display screen 30 and a housing 40 (e.g., cabinet). The 3D hologram display device 20, including the 3D hologram display screen 30 and housing 40, may vary in size and provides for universal integration and connectivity with all wireless or wired patient diagnostic devices. More particularly, the 3D hologram patient engagement system 10 is a universal device-compatible system and is designed to integrate with all handheld wireless or wired patient examination, diagnostic, and monitoring devices via USB, HDMI, Ethernet, WiFi or other forms of connectivity, as seen in FIG. 3. The 3D hologram display unit, as seen in FIG. 1, can be networked between one or more locations with encrypted connectivity for security and privacy throughout the network. As seen in FIGS. 1A and 1B, the 3D hologram display unit is housed in a cabinet 40. In one embodiment, the cabinet 40 is made of all-metal material and includes anti-radiation, as well as preventative protection from electro-magnetic or other electrical interference. In one embodiment, the 3D hologram display device is provided with an external camera 50, as well as integrated stereo speakers 60, and a touchscreen control (via the display screen 30) with integrated software. The 3D hologram display device 20 is designed to be a completely self-contained plug and play unit.

The embodiment of FIG. 1 provides a life-size transparent 3D hologram display screen 30 that uses an IPS screen, supports ultra high definition 3840×2160 4K high resolution and detailed picture quality for life-like 3D video playback (live or pre-recorded) and fully dimensional 3D graphic animations with built-in audio and video processing technology delivering an extremely high quality and engaging experience. The display screen 30 provides for superior brightness levels, ultra-wide viewing angles, high color gamut and color depth, as well as fast response image display in all light conditions.

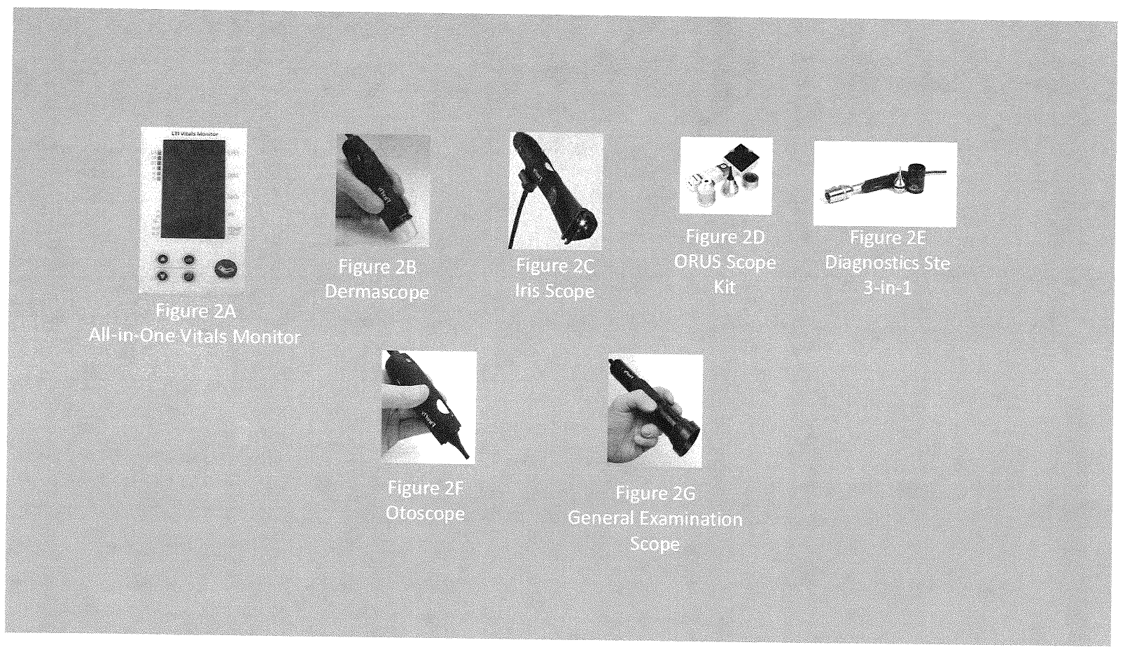

The 3D hologram patient engagement system 10 of the present invention integrates advanced technology and video processing components for superior image quality, touch-screen responsiveness, integrated media storage/server and an array of input ports (i.e., USB, HDMI, Ethernet) along with WiFi compatibility, as well as an energy saving power supply. FIGS. 2A-2G illustrate various examples of compatible devices that are able to connect either wirelessly or by wire connection to the 3D hologram patient engagement system 10. Examples of compatible devices for reference purposes include, but are not limited to: all-in-one vitals monitors; stethoscopes; general examination cameras; iris scopes; dermascopes; otoscopes; multi-purpose scope kits and other diagnostic devices now known or to become known. As seen in FIG. 3, the various compatible devices are connected to the 3D hologram display device 20 via a connectivity panel 70 inclusive of, but not limited to, HDMI, USB (all types), Ethernet and wirelessly via WiFi or other means of wireless transmission.

In one embodiment, the portable hologram display device includes the following specifications:

86 Inch 3D Hologram Display Screen

| | | |
|---|---|---|
| Transparent | 3D Transparent Display | 86″ Diagonal Display |
| Touch-screen | Size | |
| Display | 3D Transparent Display | 1895 mm (H) × 1066 mm (W) |
| Specifications | Area | 75″ (H) × 42″ (W) |
| | Display Aspect Ratio | 9:16 |
| | LCD Type | LCD panel |
| | Max Resolution | 2160 × 3840 |
| | Display “True Color” | 16.7M |
| | Brightness | White 300/150 cd/m2 |
| | Contrast Ratio | ≥1200:1 |
| | Viewing Angle | 89°/89°/89°/89° (min.) (CR ≥ 10) |
| | Response Time | 8 ms |
| | Refresh frequency | 60 Hz |
| | Color system | PAL/NTSC |
| | On-screen Menu Language | English |
| Touch-Screen | Touch Format Mode | Infrared Capacitive Touch |
| Features | Touch Points | 10 Touch Points |
| Computer Operating | Operating System | Windows |
| System & | CPU | i5 |
| Configuration | Internal Storage/RAM | 8 Gigabytes |
| | Hard Drive | 240 Gigabytes |
| Image Display | Video Formats Supported | Video Files: .wmv .avi .flv .rm .mvd |
| Video Formats | | .mpeg .ts .mp4 |
| | Audio Formats | .MP3 .WMA |
| | Picture Images Supported | BMP .JPEG .PNG .GIF |
| Integrated | HMDI | 1 |
| Connector | Ethernet | RJ45 10M/100M |
| Ports | USB | 2 |
| | Wireless Connectivity | WiFi Compatible w/ Antenna |
| Audio | Stereo Audio System | 8Ω 10 W × 2 (Optional External |
| | | Speaker Capable) |
| Power | Power Source Options | AC100 V~240 V, 50/60 HZ |
| | Maximum power | <80 W |
| | Standby power | <1 W |
| Operating | Operating Temperature | 0° C.-50° C./32° F.-122° F. |
| Environment | Range | |
| Cabinet | Frame Material: All-metal | Fabricated for Hardware & Components |
| Construction | Custom options: | Vinyl wrap, wheels, AI, selfies |
| | Interior Color: | LED backlit white display |
| | Cabinet security: | Service door lock and key |
| | Position options: | Vertical or Horizontal |

Since many modifications, variations and changes in detail can be made to the described embodiments of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A patient engagement system comprising:
a 3D hologram display unit including an exterior cabinet, and a transparent 3D hologram display screen on the cabinet for displaying a remotely located healthcare professional attending to a patient located at the 3D hologram display unit; and
a plurality of connective mechanisms for connecting patient diagnostic, examination, and monitoring devices to the 3D hologram display unit, wherein the connected diagnostic and medical examination devices are used on the patient for delivering diagnostic and other medical information concerning the patient to the healthcare professional.

2. The patient engagement system as recited in claim 1 wherein the diagnostic, examination, and monitoring devices are connected to the 3D hologram display unit via a wired connection.

3. The patient engagement system as recited in claim 2 wherein the wired connection is a USB connection.

4. The patient engagement system as recited in claim 2 wherein the wired connection is an HDMI connection.

5. The patient engagement system as recited in claim 2 wherein the wired connection is an Ethernet connection.

6. The patient engagement system as recited in claim 1 wherein the diagnostic, examination, and monitoring devices are connected to the 3D hologram display unit via a wireless connection.
